(19) United States
(12) Patent Application Publication
(10) Pub. No.: US 2018/0155675 A1
(43) Pub. Date: Jun. 7, 2018

(54) Method for Dedifferentiating and Culturing Microbial Species

(71) Applicant: Northeastern University, Boston, MA (US)

(72) Inventor: Slava EPSTEIN, Dedham, MA (US)

(21) Appl. No.: 15/575,489

(22) PCT Filed: May 23, 2016
(86) PCT No.: PCT/US16/33705

§ 371 Date: Nov. 20, 2017

Related U.S. Application Data
(60) Provisional application No. 62/164,695, filed on May 21, 2015.

(51) Int. Cl. C12N 1/36; C12M 1/34; C12N 15/01

(52) U.S. Cl. CPC C12N 1/36; C12N 15/01; C12M 41/38

(57) ABSTRACT

Methods and devices useful for reprogramming microbial cells to grow under different culture conditions are provided. The methods and devices can be used to prepare cultures of new, previously uncharacterized microbial species and for identifying laboratory conditions to culture, propagate, and study microbes that do not grow under standard laboratory conditions. The invention is also useful for characterizing microbiota, such as the communities of microorganisms inhabiting the human body and natural environments such as soil.

### BACKGROUND

It is widely known that many microbial species found in natural environments do not survive and reproduce under standard laboratory culture conditions. As a result, most such species have not been studied or exploited, even though their existence is known from sequence analysis. Nevertheless, their genes, proteins, and metabolic products represent a rich repository of biological information and potential commercial products, including antibiotics, enzymes, and pharmaceuticals. Thus, there is a need for methods of successfully culturing previously unknown microbes, especially bacteria, found in natural environments.

### SUMMARY OF THE INVENTION

The present inventor has discovered that microbes can undergo a life cycle similar to that observed in catabolic repression, where microbial cells grow under a first condition (as found in a natural environment) in which they catabolize a first nutritional substrate or collection of nutritional substrates, and are transferred to a second condition (a laboratory culture condition) in which they catabolize a second, different nutritional substrate, but only after a period of quiescence, during which they dedifferentiate and re-differentiate.

The invention employs a strategy of first rendering dormant a collection of microbial cells from a sample, such as a sample from a natural environment suspected of containing unknown microbial species. The dormant cells are then re-differentiated in a selected culture environment. After re-differentiation, the cells actively metabolize and reproduce in the selected culture environment.

Without limiting the invention to any particular mechanism, it is believed that lack of growth in culture of many microbial species is due to adaptation of the microbes to their natural environment, and successful culturing under defined laboratory conditions requires the reprogramming of microbial gene expression, which reprogramming can be accomplished by dedifferentiation to a stem cell-like state, followed by reconditioning to laboratory culture conditions.

One aspect of the invention is a method of reprogramming a population of microbial cells. In the method, one or more cells of the population of cells are incapable of reproduction in a first culture medium, and are reprogrammed to reproduce in a second culture medium. The method includes the steps of: (a) providing a sample containing the population of microbial cells; (b) applying a stress factor to the population of cells, whereby one or more of the cells become quiescent; (c) allowing the population of cells to remain quiescent for a period of time in the second culture medium; and (d) exposing the populations of cells to the first culture medium, whereby one or more of the cells that were previously incapable of reproduction in the first culture medium are reprogrammed and become capable of reproduction in the first culture medium.

Another aspect of the invention is a culture, such as a monoculture, of microbial cells in a non-naturally occurring culture medium. The culture is obtained by or obtainable by the method described above.

Yet another aspect of the invention is a device for culturing a microbial cell. The device includes a growth chamber, a port for adding or removing material to or from the growth chamber, an optional mechanism for applying a stress factor to microbial cells in the growth chamber, an optional sensor capable of detecting or analyzing metabolism or reproduction of microbial cells in the growth chamber, one or more optional fluid reservoirs, pumps, or valves for modifying or replacing a culture medium in the growth chamber, and a processor programmed to carry out the method described above.

The invention can be further summarized in the following list of embodiments.
1. A method of reprogramming a population of microbial cells, the method comprising the steps of:

(a) providing a sample comprising the population of microbial cells, the population comprising cells that are incapable of reproduction in a first culture medium;

(b) applying a stress factor to the population of cells, whereby one or more of the cells become quiescent;

(c) allowing the population of cells to remain quiescent for a period of time in a second culture medium; and

(d) exposing the populations of cells to said first culture medium, whereby one or more of the cells that were previously incapable of reproduction in the first culture medium are reprogrammed and become capable of reproduction in the first culture medium.
2. The method of embodiment 1, wherein the stress factor comprises a change in a parameter selected from the group consisting of nutrient availability, temperature, pH, ionic strength, ionic composition, availability of water, presence of waste products, exposure to a chemical agent, and exposure to radiation.
3. The method of embodiment 2, wherein the stress factor comprises exposure to a chemical agent selected from the group consisting of DNA alkylating and methylating agents, mutagens, antibiotics, metabolic poisons, enzyme inhibitors, inducers, and suppressors.
4. The method of any of embodiments 1-3, wherein the stress factor is exposure to the second culture medium.
5. The method of any of embodiment 1-4, wherein a stress factor continues to be applied during the period of time of step (c).
6. The method of any of embodiments 1-5, wherein the period of time of step (c) is hours, days, weeks, or months.
7. The method of any of embodiments 1-6, wherein a plurality of stress factors are applied, simultaneously or sequentially.
8. The method of any of embodiments 1-7, wherein step (d) comprises dividing the population of cells obtained from step (c) into portions and exposing each portion to a different first culture medium.
9. The method of any of embodiments 1-8, wherein the cell reprogramming of step (d) comprises altering the expression of one or more genes, sets of genes, polypeptides, or enzymes, or activation or inactivation of one or more metabolic pathways.
10. The method of any of embodiments 1-9, wherein the first and second culture media differ in the presence or concentration of one or more nutrients, ions, dissolved gasses, growth factors, or chemical agents.
11. The method of any of embodiments 1-10, wherein step (d) comprises a change in temperature, pH, ionic strength, or exposure to radiation compared to step (c).
12. The method of any of embodiments 1-11, wherein the reprogrammed cells form a stable culture capable of passaging in the first culture medium.
13. The method of any of embodiments 1-12, wherein the cells that become capable of reproduction in the first culture medium were not previously known to be culturable in the first culture medium.
14. The method of any of embodiments 1-13, wherein the population of cells provided in step (a) comprises previously unknown species.
15. The method of any of embodiments 1-14, wherein the sample is obtained from an environmental source or is obtained from a subject.
16. The method of any of embodiments 1-15, wherein at least some of the cells are actively growing prior to performing step (b).
17. A monoculture of microbial cells in a non-naturally occurring culture medium obtainable by the method of any of embodiments 1-16.
18. The monoculture of embodiment 17, wherein the non-naturally occurring culture medium is said first medium.
19. A device for culturing a microbial cell, the device comprising:

a growth chamber;

a port for adding or removing material to or from the growth chamber;

an optional mechanism for applying a stress factor to microbial cells in the growth chamber;

an optional sensor capable of detecting or analyzing metabolism or reproduction of microbial cells in the growth chamber;

one or more optional fluid reservoirs, pumps, or valves for modifying or replacing a culture medium in the growth chamber, and

a processor programmed to carry out the method of embodiment 1.
20. A device comprising an array of devices of embodiment 19, sharing a common processor and optionally sharing said one or more fluid reservoirs, pumps, valves, and/or mechanism for applying a stress factor.
21. The device of embodiment 19 or embodiment 20, wherein the growth chamber is fluidically connected to a second growth chamber through a nanopore.

### BRIEF DESCRIPTION OF THE DRAWINGS

FIG. 1 is a representation of three stages of the life cycle of bacteria.

FIG. 2 shows a schematic illustration of a device for reprogramming microbial cells.

### DETAILED DESCRIPTION OF THE INVENTION

The invention provides methods and devices for reprogramming microbial cells to grow under different culture conditions, especially conditions different from the native environment in which they are typically found. The methods of the invention are particularly useful for preparing cultures of new, previously uncharacterized microbial species and for identifying laboratory conditions to culture, propagate, and study microbes. The invention is also useful for characterizing microbiota, such as the communities of microorganisms inhabiting the human body and natural environments, such as soil.

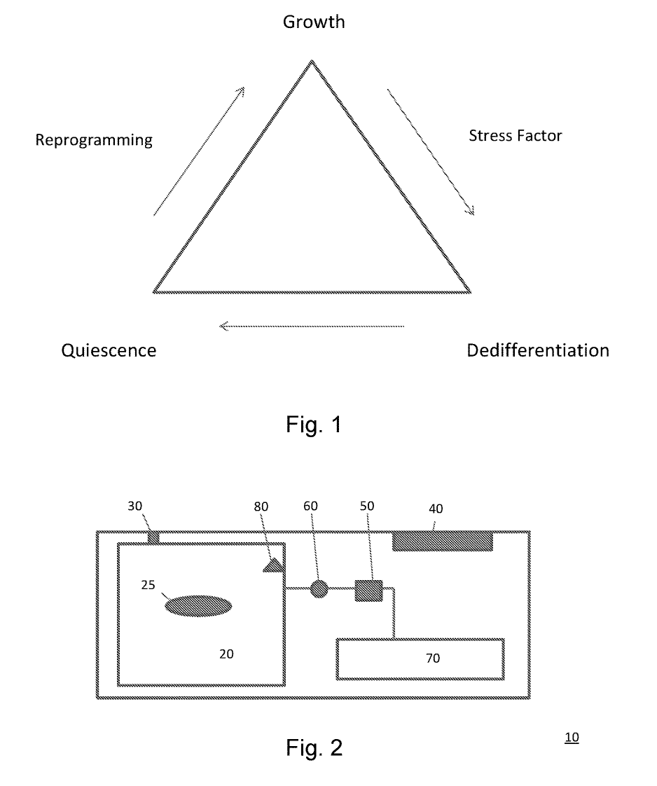

The inventor has discovered that many microbial cells, such as bacteria exhibit a life cycle that can be characterized in three stages. As shown schematically in FIG. 1, the three stages are growth, dedifferentiation, and quiescence. Microbial cells, such as bacteria, are most often found in a state of growth in their native environment, in which they have found a suitable nutrient source together with chemical and physical conditions compatible with growth and reproduction. However, when conditions become adverse to growth, brought about by a stress factor such as depletion of nutrients or appearance in the environment of chemical or physical stress, then at least some of the cells undergo a process of dedifferentiation. In the state of dedifferentiation, microbes stop growing and reproducing, alter their gene and protein expression, reduce their metabolism, and proceed into a dormant, quiescent state, such as by the formation of spores. In the state of quiescence, the cell remains dormant with reduced metabolism, waiting for its environment to become more favorable to growth. The quiescent state can continue for an extended period of time, such as hours, days, weeks, months, or even years. For a given cell in the quiescent state, the period of time spent in the quiescent state is unpredictable, and emergence back into a growth state is an essentially random, stochastic process. During or at the end of the dormant stage, the cell undergoes reprogramming, or the alteration of gene expression, resulting in modified metabolism and ability to re-enter the growth state. The resumption of growth depends on the cell resuming gene and protein expression under suitable environmental conditions consistent with active metabolism, growth and reproduction.

To practice the method, a sample suspected of containing microbial cells is obtained. The method can be used with samples containing, for example, eubacteria, archaebacteria, or other microorganisms such as, e.g., microalgae, or fungi, such as yeast. The method can be practiced with either prokaryotic or eukaryotic microorganisms. The sample can be from any source. Preferably the sample is harvested from an environment containing a community of microbial cells and suspected of containing cells of interest that do not grow under typical or known culture conditions. For example, the environment can be a natural environment, such as a body of water, soil, rock, or air. The sample also can be obtained from a plant or animal, such as a human, including from the human gut microflora, from blood, urine, or other bodily fluid, or from a wound, wound fluid, or an infected tissue. Samples also can be harvested from a surface or other portion of a material suspected of harboring microbial cells of interest. The sample can be optionally processed by dilution, centrifugation, filtration, addition of growth media, nutrients, or other chemical substances. Optionally, the sample can be used without processing or any modification, but retained in its natural milieu, though separated or isolated from its original environment. The sample can be treated to select for or eliminate certain classes of cells, such as growing or reproducing cells, non-growing or non-reproducing cells, fast growing or slow growing cells, spore forming cells, cells having certain morphology or size, cells having certain metabolic or nutrient preferences, cells having or lacking certain antibiotic sensitivity, and the like. The sample can also be analyzed to genetically identify cells present in the sample, such as by 16S rRNA sequencing.

The sample is then subjected to a physical or chemical stress factor that leads to dedifferentiation. Suitable stress factors can include starvation (e.g., by deprivation from fresh nutrients, air, water, or light), heat, cold, desiccation, addition of chemicals (e.g., mutagens, toxins, DNA-disrupting agents or alkylating agents), or alteration of pH, ionic strength, or osmolarity, or any combination thereof. In certain embodiments, the use of high concentrations of nutrients is avoided as a stress factor. The period of stress can be in the range of, for example, 1, 2, 3, 5, 8, 12, or 24 hours, or 1, 2, 3, 5, 10, 14, or 30 days, or 1, 2, 3, 4, 5, 6, 8, 10, 12, or 16 weeks. Preferably, the period of stress is maintained for 1-2 weeks. In certain embodiments, the stress factor or factors are applied to microbial cells individually, with a single cell per chamber, isolated from other cells by a wall or membrane; in such an arrangement, different stress factors and/or combinations thereof can be tested simultaneously. For example, cells from the originally provided sample can be diluted in a suitable medium and placed into an array of chambers, such as in a microfluidic device, with on average one cell per chamber. Each chamber can have an inlet/outlet port, or separate inlet and outlet ports, which allow the culture medium to be replaced, exchanged, or modified, such as by addition of chemical substances and nutrients. This approach can be used to obtain monocultures of microbial species, including newly discovered or previously unknown species. Alternatively, a mixture of different cells can be used, in which case a mixed culture can be obtained, although a monoculture also can be obtained if conditions are found that stimulate the growth of a single species.

In practicing a method of the invention, one or more stress factors are allowed to remain in place for a period of time. Either one or more than one stress factor can be applied, either simultaneously or in any desired sequence. If two or more stress factors are applied serially, they can either be applied without a pause between them, or with an interval of rest between them. Dedifferentiation is eventually induced by exposure to stress. Such dedifferentiation is characterized by a change in gene expression, wherein the expression level of one or more genes is altered, particularly those regulating cellular metabolic pathways, and cell structure may also change. The microbial cells become quiescent as a result of dedifferentiation. Their metabolic activity slows or comes to a halt, and cell division ceases. At the end of the period of dedifferentiation, the cells enter the state of quiescence.

Cell dedifferentiation and exposure to one or more stress factors can take place in a liquid culture medium or on the surface of a solid medium, such as agar. Once the microbial cells have become dedifferentiated and exposure to stress factors is no longer required, they optionally can be transferred into a different culture chamber containing a different selected culture medium. In certain embodiments, the culture medium containing the dedifferentiated cells can be diluted or small volumes distributed into a plurality of individual growth chambers, preferably such that each growth chamber contains no more than a single microbial cell. In certain embodiments, the cells are distributed into such individual growth chambers prior to the application of stress, and remain in those chambers through their period of quiescence. The growth chambers are maintained under conditions suspected of promoting eventual reprogramming and activation of the microbial cells by return to a growth state. The growth chambers can have a transparent portion, such as the chamber bottom or a window, to allow visual and/or microscopic inspection. A plurality of growth chambers can be arranged into an array, such as a standard microtiter plate, for convenient manipulation.

The term “culture medium” or “growth medium” as used herein refers to any liquid, semi-solid (e.g., gel), or solid medium on which or in which microbial cells can survive, grow, metabolize, and/or reproduce. A liquid culture medium is typically water based, and can be pure water or water containing one or more electrolytes and one or more nutrients. The culture medium can be a nutrient solution or gel such as a standard liquid bacterial growth medium or agar-based culture medium in a plate or well. The culture medium can be supplemented with desired nutrients, such as amino acids, sugars, nucleic acids, antibiotics, growth factors, or other chemicals. The culture medium, growth chamber, and all chemical agents added to the growth chamber are initially sterile and handled so as to avoid contamination. The growth chamber can be monitored at intervals so as to determine when the microbial cell or cells have become activated, and growth and reproduction resume. Monitoring can also be carried out remotely with the aid of one or more sensors that can signal when metabolism, growth, or reproduction has resumed.

Once cells have entered the quiescent state, they are allowed to remain in that state fora period of time, such as 1, 2, 3, 5, 8, 12, or 24 hours, or 1, 2, 3, 5, 10, 14, or 30 days, or 1, 2, 3, 4, 5, 6, 8. 10, 12, or 16 weeks, or 1, 2, 3, 4, 5, or 6 months, or 1, 2, or 3 years or longer. Preferably the cells are allowed to remain in the quiescent state for a long period of time, such as 2, 4, 6, 8, 12, or 16 weeks, or for 1, 2, 4, 6, 8, 10, or 12 months. During the quiescent period, the culture medium can be exchanged or processed to add nutrients or remove waste products during the incubation period.

An important objective of the methods of the invention is to provide a culture of microbial cells that are actively growing and reproducing in a culture medium, so that they can be propagated and studied, and so that stable cell lines can be preserved and used to produce commercial products, such as antibiotics, as well as other useful chemical products and biomolecules. The major impediment to this, which is overcome by the present invention, is the lack of a known culture medium and other culture conditions, such as temperature, pH, chemical milieu, presence or absence of oxygen, etc., in which a given microbial cell type can be maintained and propagated. Thus, of interest is the propagation of microbial cells that initially cannot be propagated in a first culture medium, and under first culture conditions, but through a method of the invention are reprogrammed to live and propagate in the first culture medium, and/or under the first culture conditions. Since the cells are initially incapable of propagation in the first culture medium, they are initially maintained, optionally during the exposure to one or more stress factors, but in particular during the quiescent phase, in a second medium. In certain embodiments, exposure to the first medium can be used as the stress factor; in other embodiments, exposure to the first medium is avoided prior to establishing quiescence and the first medium is not used as a stress factor. The cells are capable of survival in the second medium, but incapable of propagation. During or following incubation in the second medium, the cells are introduced to the first culture medium, and/or to the first culture conditions. Introduction to the first medium can be gradual or sudden. Gradual conversion to first medium can be performed in different ways. For example, the first medium can be mixed with the second medium, e.g., in gradually increasing amounts, or the second medium can be replaced completely with first medium for a brief interval, followed by return to second medium. Such intervals can be increased gradually over time, until the cells are able to remain in the first medium. Another way to gradually convert from second to first medium is to add one or more individual components or conditions of the first medium to the second medium, until the second medium is substantially converted into the first medium or conditions.

During the incubation period in the quiescent phase, one or more of the cells undergoes genetic reprogramming resulting in acquisition of the ability to survive and propagate in the first medium. Without intending to limit the invention to a particular mechanism, the awakening from the quiescent state is believed to result from a stochastic process, in which individual cells activate the expression of certain genes or collections of genes randomly, allowing them to collectively probe their environment for nutrients and conditions that support their growth and reproduction.

Once the quiescent cells are exposed to first medium, or first culture conditions, the cells are observed for signs of increased metabolic activity or cell reproduction. Observation can be visual, with aid of a microscope, in which case cell size, morphology, and number can reveal when the cells have been reprogrammed and are capable of growth and reproduction. Stains, immunoreagents, polymerase chain reaction, and other techniques also can be used to assess changes in metabolic activity or protein expression; such methods can be carried out as operator-assisted assays or can be realized through the presence of one or more sensors present in the cell culture chamber. Monitoring of cell awakening can be entirely automated as well, such as in a microfluidic embodiment.

Once actively dividing cells have been obtained, conventional microbiological techniques can be utilized to maintain and further investigate the culture. For example, cells can be passaged, cryopreserved and frozen, or subcultured to obtain genetically homogeneous monocultures. Co-cultures containing two or more species or varieties of microbial cells can also be obtained. Monocultures of a single microbial species and co-cultures containing two or more species are products of the invention; many such monocultures, co-cultures, or even communities of microorganisms that are actively growing and reproducing in newly identified culture media and culture conditions will be novel and unique, often representing previously unknown microbial species, or species that were previously identified but could not be cultured under conditions allowing them to actively metabolize, grow, reproduce, or produce useful products without applying a method or device of the invention.

In a method of the invention, a plurality of individual cells, simultaneously and in parallel, are exposed to one or more stress factors, dedifferentiated, rendered quiescent, and then induced to re-differentiate and become metabolically and/or reproductively active. Parallel reprogramming of microbial cells can be carried out manually, using conventional culture dishes or tubes filed with culture media. It also can be carried out using a set of individual devices, such as fluidic or microfluidic devices, one for each sample (group of cells) or starting cell (after suitable dilution). However, parallel reprogramming also can be accomplished using one or more single devices (e.g., microfluidic devices), each containing an array of growth chambers in a single device. Such a device can be used in different ways. In one embodiment of using such a device, the device contains an array of growth chambers, and each chamber is filled with the same culture medium and is treated in the same manner. For example, cells in each chamber are exposed to the same stress factor(s) at the same time, and converted to the first culture medium in the same manner and at the same time. The cells of a single sample or a plurality of samples are distributed into the chambers, one or more cells to a chamber. The use of a single cell per chamber has significant advantages, in particular that each cell has the potential to produce a monoculture of a single species. In another embodiment, the culture medium, stress factor(s), and/or method of treating the cells to induce reprogramming can vary across the array of the device. In this manner, more conditions can be tried out for a given sample or cell type at one time, and the time to discovery of an appropriate culture medium and culture conditions can be shortened dramatically.

The invention further contemplates a device for dedifferentiation and re-differentiation of a microbial cell. An embodiment of such a device is depicted in FIG. 2 (structures are not shown to scale). Device 10 contains one or more growth chambers 20 containing one or more microbial cell 25, one or more ports 30, and processor 40 which can be programmed to carry out a method of the invention. The device can optionally include a mechanism for applying one or more stress factors to microbial cells in the growth chamber; such a device can be a device for heating and/or cooling the chamber (not shown), a radiation source (not shown), or pump 50, valve 60, and one or more fluid reservoirs 70 containing chemical agents for delivery to the growth chamber to act as stress factors. The same or different pumps, valves, and fluid reservoirs can be used to exchange or modify the composition of culture medium in the growth chamber. An additional optional component is one or more sensors 80, which detect changed conditions within the growth chamber, such as the number of cells, presence or concentration of a metabolite, presence or concentration of a nutrient, presence or amount of specific cell proteins, antigens, or nucleic acids, and the like. Yet another optional component is an optical window for monitoring cell condition, growth, and/or number, so as to detect signs of reprogramming or reproduction. Further optional components include a memory connected to the processor and/or sensors for data storage, a wireless transmitter to communicate data from the device to a remote monitoring station or user's cell phone or computer and receiver for receiving user instructions or modified programming, and a display to provide a readout of data or conditions of the device or within a growth chamber or reservoir of the device. In certain embodiments, the device includes an array of growth chambers, such as 2, 4, 10, 100, 1000, 10000, or 1000000 or more chambers on a single device, or can use a microtiter plate format, such as 6, 24, 96, 384, 1536, 3456, or 9600 wells on a single device. In certain embodiments, each growth chamber can be fluidically coupled to an adjacent growth chamber, such as one filled with the first culture medium. The fluid pathway joining the adjacent growth chambers contains a constriction small enough to allow only a single cell to enter the adjacent chamber, such as by proliferating within the fluid pathway. The fluid pathway joining the chambers can be, for example, a microfluidic or nanofluidic channel, such as one having a diameter in the range from about 500 nanometers to about 1500 nanometers, and a length from about 1 to about 10 microns. See, for example, US Appl. No. 2015/0167043.

This application claims the priority of U.S. Provisional Application No. 62/164,695 filed 21 May 2015 and entitled “Method for Dedifferentiating and Culturing Microbial Species”, the whole of which is hereby incorporated by reference. The following US patents or printed publications are also incorporated by reference in their entireties: US Appl. No. 2015/0167043, U.S. Pat. No. 7,011,957, and U.S. Pat. No. 9,249,832.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

1. A method of reprogramming a population of microbial cells, the method comprising the steps of:
(a) providing a sample comprising the population of microbial cells, the population comprising cells that are incapable of reproduction in a first culture medium;
(b) applying a stress factor to the population of cells, whereby one or more of the cells become quiescent;
(c) allowing the population of cells to remain quiescent for a period of time in a second culture medium; and
(d) exposing the populations of cells to said first culture medium, whereby one or more of the cells that were previously incapable of reproduction in the first culture medium are reprogrammed and become capable of reproduction in the first culture medium.

2. The method of claim 1, wherein the stress factor comprises a change in a parameter selected from the group consisting of nutrient availability, temperature, pH, ionic strength, ionic composition, presence of waste products, availability of water, exposure to chemical agents, and exposure to radiation.

3. The method of claim 2, wherein the stress factor comprises exposure to a chemical agent selected from the group consisting of DNA alkylating and methylating agents, mutagens, antibiotics, metabolic poisons, enzyme inhibitors, inducers, and suppressors.

4. The method of claim 1, wherein the stress factor is exposure to the second culture medium.

5. The method of claim 1, wherein a stress factor continues to be applied during the period of time of step (c).

6. The method of claim 1, wherein the period of time of step (c) is hours, days, weeks, or months.

7. The method of claim 1, wherein a plurality of stress factors are applied, simultaneously or sequentially.

8. The method of claim 1, wherein step (d) comprises dividing the population of cells obtained from step (c) into portions and exposing each portion to a different first culture medium.

9. The method of claim 1, wherein the cell reprogramming of step (d) comprises altering the expression of one or more genes, sets of genes, polypeptides, or enzymes, or activation or inactivation of one or more metabolic pathways.

10. The method of claim 1, wherein the first and second culture media differ in the presence or concentration of one or more nutrients, ions, dissolved gasses, growth factors, or chemical agents.

11. The method of claim 1, wherein step (d) comprises a change in temperature, pH, ionic strength, or exposure to radiation compared to step (c).

12. The method of claim 1, wherein the reprogrammed cells form a stable culture capable of passaging in the first culture medium.

13. The method of claim 1, wherein the cells that become capable of reproduction in the first culture medium were not previously known to be culturable in the first culture medium.

14. The method of claim 1, wherein the population of cells provided in step (a) comprises previously unknown species.

15. The method of claim 1, wherein the sample is obtained from an environmental source or is obtained from a subject.

16. A monoculture of microbial cells in a non-naturally occurring culture medium obtainable by the method of claim 1.

17. The monoculture of claim 16, wherein the non-naturally occurring culture medium is said first medium.

18. A device for culturing a microbial cell, the device comprising:
a growth chamber;
a port for adding or removing material to or from the growth chamber;
an optional mechanism for applying a stress factor to microbial cells in the growth chamber;
an optional sensor capable of detecting or analyzing metabolism or reproduction of microbial cells in the growth chamber;
one or more optional fluid reservoirs, pumps, or valves for modifying or replacing a culture medium in the growth chamber, and
a processor programmed to carry out the method of claim 1.

19. A device comprising an array of devices of claim 18, sharing a common processor and optionally sharing said one or more fluid reservoirs, pumps, valves, and/or mechanism for applying a stress factor.

20. The device of claim 18, wherein the growth chamber is fluidically connected to a second growth chamber through a nanopore.
